(19) United States
(12) Patent Application Publication
(10) Pub. No.: US 2015/0100090 A1
(43) Pub. Date: Apr. 9, 2015

(54) IMPLANTABLE APPARATUS FOR MODULATION OF SKELETAL GROWTH

(71) Applicant: John Burke, Edinburgh (GB)

(72) Inventor: John Burke, Edinburgh (GB)

(21) Appl. No.: 14/520,834

(22) Filed: Oct. 22, 2014

Related U.S. Application Data
(63) Continuation-in-part of application No. 12/450,950, filed on Oct. 19, 2009, now abandoned.

(30) Foreign Application Priority Data
Apr. 17, 2007 (GB) 0707285.3

(51) Int. Cl. A61B 17/70

(52) U.S. Cl. CPC A61B 17/7061
USPC 606/257

(57) ABSTRACT

An implantable apparatus for use in the correction of skeletal deformities comprising a hinge applied in various orientations across one or more growth plates and attached to bone via attachment members.

### CROSS-REFERENCE TO RELATED APPLICATION(S)

This application is a continuation-in-part of U.S. application Ser. No. 12/450,950 filed Oct. 19, 2009 for “Implantable Apparatus For Modulation Of Skeletal Growth” by J. Burke, which in turn is the national phase of PCT Application No. PCT/GB2008/050269 filed Apr. 17, 2008 for “Implantable Apparatus For Modulation Of Skeletal Growth” by J. Burke, which in turn claims priority from British Application No. 0707285.3 filed Apr. 17, 2007.

### INCORPORATION BY REFERENCE

U.S. application Ser. No. 12/450,950, PCT Application No. PCT/GB2008/050269 and British Application No. 0707285.3 are hereby incorporated by reference in their entirety.

### FIELD OF THE INVENTION

The invention relates to apparatus for correcting skeletal deformities, and in particular to an implantable device for controlling the relationship of bones on either side of a growth plate in the skeletally immature. It also has an application for dynamic skeletal stabilisation in the adult and in some embodiments as a dynamic compression plate for fracture treatment.

### BACKGROUND OF THE INVENTION

A number of conditions give rise to paediatric deformity, for example scoliosis (curvature of the spine), club foot, post traumatic deformity such as cubitus varus, genu varum and valgum, cerebral palsy (where abnormal muscles forces result in asymmetric loading of the growth plates with subsequent development of deformity), developmental dysplasia of the hip and perthes disease in addition to numerous other less common conditions.

Scoliosis is one of the principal skeletal disorders that this device has been devised to correct. Scoliosis can be divided into early and late onset types according to the age at which it develops. Factors for the development of early onset scoliosis vary considerably and include idiopathic and neuromuscular aetiologies.

Early onset scoliosis is currently managed conservatively with the use of casts and braces to delay curve progression until the child is old enough for definitive treatment by instrumentation and fusion of the curve. Where conservative therapy fails, a variety of surgical procedures are used. For example, convex epiphysiodesis, stapling of the convex side of the curve, short segment fusion, and posterior growing rod systems such as the Harrigton rod, ISOLA® growing rods or the Luque trolley system.

The Luque trolley system, described in French published patent application No 2589716 comprises a pair of U-shaped callipers fixed to the spine, one of the callipers sliding within the other so that the spine may grow. One problem with this system is that the vertebrae of the spine may fuse spontaneously as a result of exposing the spine to fit the callipers.

Using the ISOLA® system, rods are inserted in a way that causes fusion of a small segment of spine proximally and distally to give a solid anchor for the rods. The rods do not control the sagittal profile adequately and obtain their correction solely by distraction. In order to lengthen the rods to accommodate growth, the patient must be operated on every six months or so. Rod breakage is a frequent occurrence and to reduce this risk most patients are also treated in a brace to reduce the implant stresses. The rods need to be changed when they get too short necessitating a somewhat larger surgical procedure than a standard rod lengthening procedure. The purpose of the pediatric ISOLA® is to stabilize the deformity and allow some growth to occur. Definitive treatment of the scoliosis usually involves a further operation where vertebrae are fused together. Insertion of posterior growing rods and the final fusion procedure are relatively major surgical procedures especially if a thoracotomy is required for anterior release of the curve prior to insertion of the growing rods. The treatment regime for progressive early onset scoliosis as outlined above is arduous. It has a profound impact on the child's biopsychosocial profile. There is thus a great need for better treatment options in this patient group.

Growth plates are affected by mechanical stimuli. Increased pressure on a growth plate reduces growth rate, whilst decreased pressure on a growth plate or traction can increase growth rate (Heuter Volkmann Law).

Once a scoliosis curve develops the biomechanics of the spine are altered resulting in compression of the concave side of the curve and decreased pressure on the convex side of the curve. Growth on the concave side of the curve is therefore retarded whilst growth on the convex side is accelerated. The spine grows asymmetrically and the deformity is exacerbated. The present invention utilises the Heuter Volkmann Law to reverse the deformity as growth occurs.

The Heuter Volkmann law is an oversimplification of the actual relationship of growth to mechanical stimuli. There is evidence that mechanical stimulation/pressure increases growth up to a point beyond which it prevents growth. Decreased mechanical stimulation/pressure across a growth plate below the maximal stimulation level decreases growth. Tension across a growth plate increases the growth rate but not as much as optimal pressure. Furthermore one has to consider the effect of static forces across a growth plate in comparison to dynamic forces as they have different effects. Our understanding of the mechanical modulation of growth is in its infancy. It suffices to say that we know that it is possible to alter growth by mechanical stimulation but that it is difficult to predict accurately exactly what effect a particular mechanical stimulus will have.

Adolescent idiopathic scoliosis (AIS) develops during the adolescent growth spurt and is the predominant form of late onset scoliosis. It is currently managed by observation, bracing or fusion, the goals of management being to prevent further deterioration in the condition and get some correction of the deformity. The difficulty in managing AIS lies in the absence of any non-surgical treatment which can alter the natural history of the disease. Observation involves watching the curve get worse until bracing and/or surgical intervention can be justified. Bracing has been shown to be largely ineffective although it is still widely practised. Many curves do not progress to an extent where surgical intervention is required but at the present time there is no way of determining accurately which curves will progress although work is ongoing to make prediction of curve progression more accurate. The current surgical treatment for scoliosis consists of fusion of the curve with partial correction of the deformity. This is a major undertaking and so not justified for curves under 40 degrees.

Recently there has been considerable interest in non fusion treatments for scoliosis. To date these treatments have involved stapling or tethering the convex aspect of the curve and although these efforts have met with some success in preventing curve progression they have not been able to correct the deformity. There is a need for a device which can prevent progression and reverse the deforming process in scoliosis allowing scoliosis to be managed proactively and without the need for major surgical intervention in the form of spinal fusion.

The current management strategy in late onset scoliosis involves observing and bracing a progressive curve until it progresses sufficiently with the patient's growth to warrant very major surgical intervention in the form of spinal fusion. The patient's residual growth potential which might be used to correct the deformity is thus squandered and instead acts to perpetuate the curve.

The presence of a non fusion device on the market which could be surgically implanted by a minimally invasive technique and which halts the deforming process and starts growing the spine back towards a normal shape would be an enormous advance in this area. Such a device would be safer still if it did not require the spinal segmental blood vessels to be sacrificed and did not involve moving the position of the spine as it was applied. These latter two considerations greatly reduce the risk of any neurologic impairment arising from the surgery. This type of procedure could be used instead of bracing in those who were not compliant or in those cases where it was considered that there was a high risk of curve progression (those with high residual growth potential). It is precisely the latter group of patients who can be most effectively treated with this type of device.

Another factor which may be important in the long term outcome of non fusion surgical therapies for scoliosis is whether movement can occur at the disc space during the treatment.

Rigid fixation has been shown to cause disc degeneration and would therefore exacerbate the disc abnormalities already present in the scoliotic spine segment. Therefore an implant which allows sufficient motion to keep the tissues healthy is desirable. Moreover application of this device earlier in the disease should help reduce the amount of disc degeneration occurring in the curve. This would have the added benefit of reducing the stresses on the implant thus reducing the risk of mechanical failure.

A further consideration in a growth modulating implant is whether it loads/unloads the growth plate dynamically or statically. There is some evidence to suggest that dynamic loading stimulates growth more effectively than static loading although the extent of load and optimum frequency of loading to maximally stimulate growth remain unknown although work is currently underway to define these parameters.

High residual growth potential (HRGP) is the most important prognostic indicator in determining the likelihood of progression of scoliosis. Hence HRGP is currently regarded as a bad thing by those managing scoliosis in the skeletally immature.

The dilemma in treating these patients is that it is necessary to allow growth to occur to avoid cardiorespiratory compromise but necessary to prevent curve progression as this will ultimately compromise cardiorespiratory function more than if no growth was permitted.

The present invention allows HRGP to be controlled and used to correct the spinal deformity. It represents a new departure in the management of scoliosis in the skeletally immature and demands a change in attitude towards HRGP by those treating this condition. The device has turned HRGP into an asset in the management of scoliosis. Hence a new philosophy is required by those treating scoliosis in the skeletally immature, in which HRGP is regarded as a good thing and utilised to correct the deformity.

The present invention provides a partial correction of the deformity at the time of application (spinal curvature partly corrects spontaneously with the patient lying on the operating table). However with growth it acts to gradually correct the spinal deformity which is theoretically safer on the neurological structures than the multiple acute corrections obtained by repeated surgical manipulation of current growing rod constructs. The device acts to gradually derotate the spine and the rib cage theoretically allowing restoration of normal respiratory mechanics and eliminating the rib hump. The reversal of the initial deforming forces, by the implant will promote remodelling of the ribs in addition to the mechanical derotation that occurs with growth. This combination of rib derotation and remodelling would be expected to restore the rib cage to near normal, eliminating the rib hump which is usually the major cause of cosmetic deformity. No other treatment method offers the potential for comprehensive restoration of respiratory mechanics and complete elimination of the rib hump and the rib hollow on the concave side.

The device takes us into a new era in scoliosis surgery where high residual growth potential for the first time becomes an advantage in the treatment of spinal deformity. The implant may be inserted by a minimally invasive approach, which combined with its non-fusion technology takes scoliosis treatment in the skeletally immature to new levels.

It would be desirable to provide an improved implantable apparatus for correcting skeletal deformities.

### SUMMARY OF THE INVENTION

The invention provides an implantable device for use in the correction of skeletal deformities, which allows movement of bones during growth.

In a preferred embodiment the implantable apparatus comprises at least one attachment member for attaching the apparatus to a bone such as a vertebra and at least one hinge element to allow movement of the attachment members.

In a preferred embodiment the hinged element connects one or more attachment members which attach to a bone on either side of a growth plate.

Preferably, the attachment members may be bone screws and/or blade plates.

The attachment members may be attached to the hinge element or formed integrally with the hinged element.

The hinge element may comprise a hinge of any known type.

The hinge element may comprise a sloppy hinge, allowing motion to a variable extent in two or three dimensions.

The hinge element may have coupled motion incorporated into its structure so as the hinge opens the movement forces another desired movement of a hinge component.

The hinge element may be in a rising butt configuration to allow rotation to occur at a point distant from the hinge.

The implantable device of the invention may be orientated in a variety of different ways to treat different skeletal deformities.

The hinge element of the device may be orientated in different predetermined ways with respect to the attachment members and/or to a growth plate in a patient to be treated.

The hinge element may allow movement to occur in three dimensions at a hinged bearing.

The orientation of the device when fitted to a patient may restrict or encourage movement and or growth of skeletal structures in a particular direction.

The implantable device may have one or more constraints of movement incorporated into its structure.

In one embodiment the device may include an adjustment element such as a screw to alter the position of a constraint in the device. Such adjustment may be made after the application of the device to a patient.

The implantable device may have one or more dynamic elements incorporated into its structure such as a biasing means, which may be in the form of a spring, or other device. The biasing means may comprise a spring, such as a coil spring or leaf spring arranged within the hinge, or between the hinge limbs. By arranging the biasing means within the hinge, it is possible to keep the biasing means away from contact with body tissue.

Advantageously, a number of the implantable devices can be used on a single patient and the devices may be connected together to form a single construct.

The implantable device may be connected to other devices by a structure or cable to prevent loss of control of a deformity if one device fails structurally.

In use, the device may be introduced to a patient in a manner similar to a staple.

The device may be used in other surgical procedures and for purposes other than the correction of spinal deformities.

In one embodiment, the implantable device may have more than one hinge element between attachment members.

In one embodiment, the implantable device may have more than one hinge arranged at an angle to each other.

The device may be used to correct spinal deformity, other skeletal deformities, as a dynamic stabiliser or as a dynamic compression plate.

The device may be applied to the spine of a patient by anterior or posterior surgical approaches.

A further aspect of the invention may comprise a kit of implantable devices and/or hinge elements.

The device may be provided in a range of sizes and configurations, depending on the specific deformity to be treated and the patient size.

### BRIEF DESCRIPTION OF THE DRAWINGS

FIG. 1 shows an anterior view of a spinal motion segment in a scoliosis deformity with the implantable apparatus attached to the convex side of the scoliosis.

FIG. 2 shows a lateral view of a spinal motion segment in a scoliosis deformity with the implantable apparatus of FIG. 1 attached to the convex side of the scoliosis.

FIG. 3 shows a superior view of a spinal motion segment in a scoliosis deformity with the implantable apparatus of FIG. 1 attached to the convex side of the scoliosis.

FIG. 4 shows an anterior view of a number of spinal motion segments in a scoliosis deformity with a number of the implantable devices of FIG. 1 applied to the convex aspect of the curve.

FIG. 5 shows an anterior view of the instrumented spinal deformity of FIG. 2a after a period of time when sufficient growth has occurred to correct the spinal deformity.

FIG. 6 shows a superior view of a spinal motion segment in a scoliosis deformity with the hinge of the implantable apparatus of FIG. 1 parallel to the anteroposterior axis of the spine.

FIG. 7 shows a superior view of a spinal motion segment in a scoliosis deformity with the hinge of the implantable apparatus of FIG. 1 angled anteriorly to the anteroposterior axis of the spine.

FIG. 8 shows a superior view of a spinal motion segment in a scoliosis deformity with the hinge of the implantable apparatus of FIG. 1 angled posteriorly to the anteroposterior axis of the spine.

FIG. 9 shows an anterior view of a spinal motion segment in a scoliosis deformity with the implantable apparatus of FIG. 1 attached to the convex side of the scoliosis.

FIG. 10 shows a lateral view of a spinal motion segment in a scoliosis deformity with the implantable apparatus of FIG. 1 attached on the lateral aspect of the spine.

FIG. 11 shows a superior view of a spinal motion segment in a scoliosis deformity with the implantable apparatus of FIG. 1 attached on the lateral aspect of the spine.

FIG. 12 shows a coronal cross-section of one configuration of the hinge bearing.

FIG. 13 shows a cross-section of the hinge bearing in the sagittal plane.

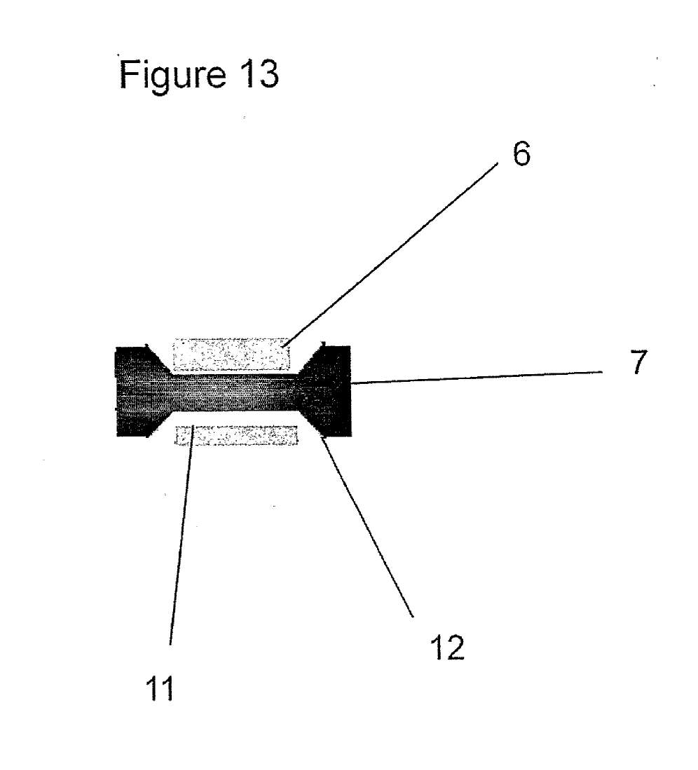

FIG. 14 shows a lateral view of a spinal motion segment in a scoliosis deformity with an embodiment of the device attached to the lateral aspect of the spine on the convexity of the curve.

FIG. 15 shows a lateral view of a spinal motion segment in a scoliosis deformity with an embodiment of the device attached to the lateral aspect of the spine.

FIG. 16 shows a lateral view of a spinal motion segment in a scoliosis deformity with an embodiment of the device attached to the lateral aspect of the spine on the convexity of the curve.

FIG. 17 shows a superior view of a spinal motion segment in a scoliosis deformity with an embodiment of the device is attached to the lateral aspect of the spine on the convexity of the curve.

FIG. 18 shows a superior view of a spinal motion segment in a scoliosis deformity with an embodiment of the device attached to the lateral aspect of the spine on the convexity of the curve.

FIG. 19 shows an anterior view of a spinal motion segment in a scoliosis deformity.

FIG. 20 shows a hinge viewed from the anterior aspect.

FIG. 21 shows a hinge viewed from the anterior aspect and illustrates the effect of initial hinge position on amount of longitudinal growth permitted by the device.

FIG. 22 shows a hinge viewed from the anterior aspect and illustrates the effect of initial hinge position on amount of longitudinal growth permitted by the device.

FIG. 23 shows an anterior view of a spinal motion segment in a scoliosis deformity with an embodiment of the hinge device attached on the curve convexity.

FIG. 24 shows an anterior view of a spinal motion segment in a scoliosis deformity with an embodiment of the hinge device attached on the concavity of the curve.

FIG. 25 shows a hinge viewed from the anterior aspect and illustrates the effect of initial hinge position on amount of longitudinal growth permitted by the device.

FIG. 26 shows a schematic representation of the hinge of the implantable device.

FIG. 27 shows a schematic representation of the implantable apparatus incorporating more than one implantable device.

FIG. 28 shows a schematic representation of an alternative embodiment of the implantable device, having a ball and socket type hinge.

### DETAILED DESCRIPTION OF THE PREFERRED EMBODIMENTS

The present invention provides various embodiments of implantable apparatus and methods for controlling spinal growth in scoliosis and other spinal deformities. The devices act to prevent growth which would result in a worsening of the deformity and utilise growth to correct the spinal deformity. These devices have a broader application and may be used to treat other skeletal deformities and may have other surgical or non-surgical uses. The device may be used as a dynamic spine stabiliser in adults with spinal deformity.

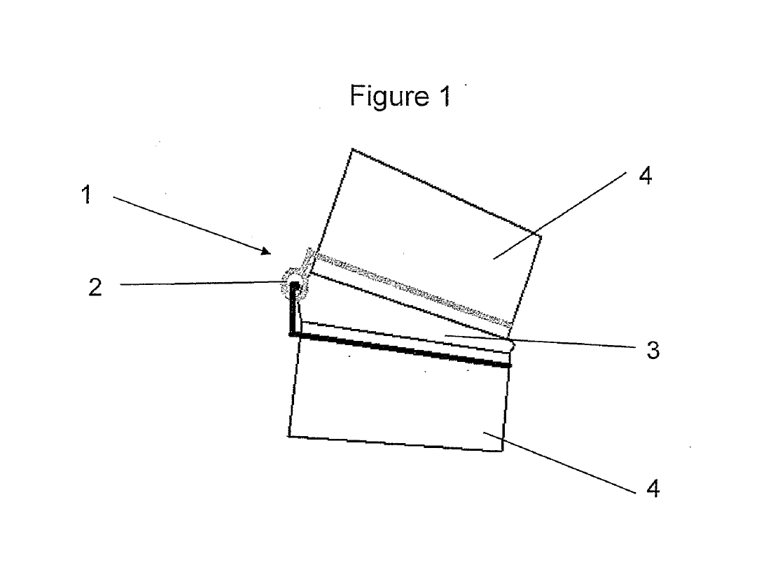

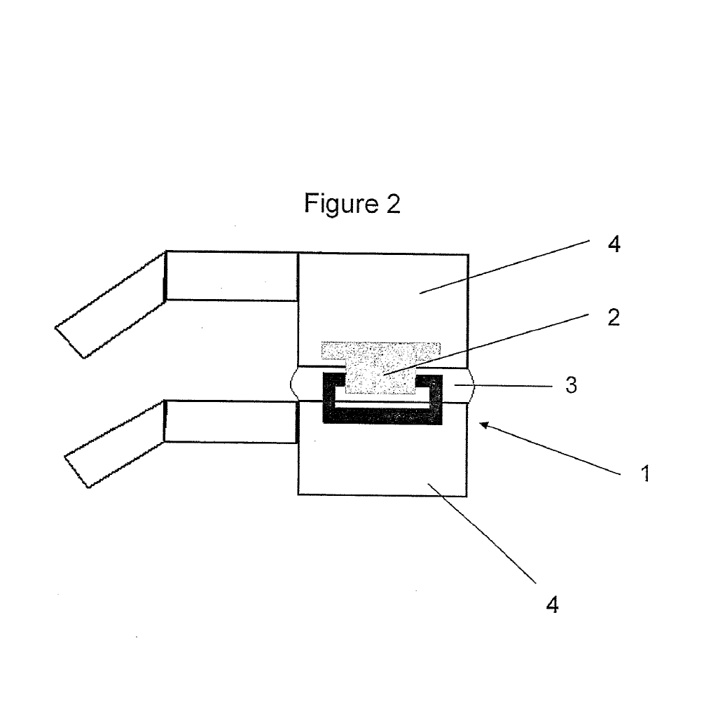

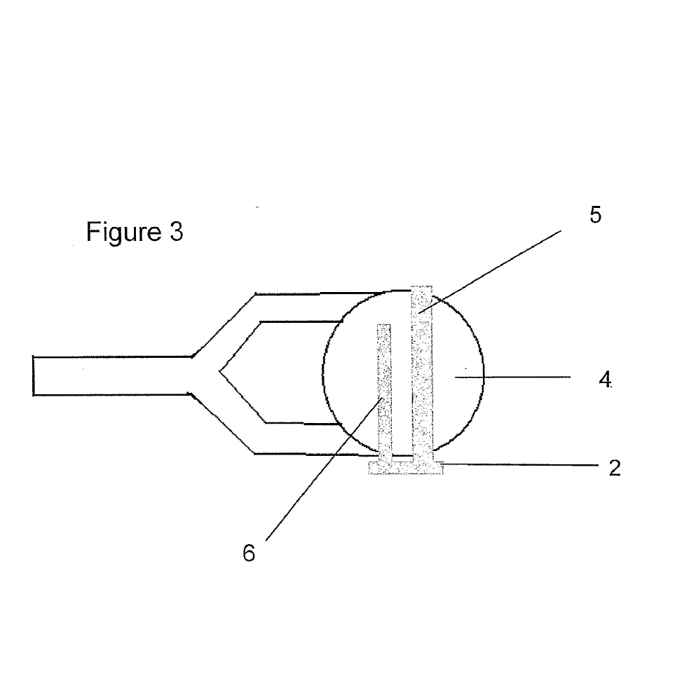

As illustrated in FIGS. 1 to 3, in a preferred embodiment the device 1 includes a hinge 2 which is applied across a growth plate or intervertebral disc 3 between two vertebral bodies 4.

FIG. 1 shows the device 1 applied on the convex side of a scoliosis or spinal curve. Attachment members 5, 6 are attached to the vertebral body 4 above and below the growth plate and intervertebral disc 3. The device 1 therefore spans the growth plate and intervertebral disc 3 and the hinge 2 tethers the convex aspect of the curve and as growth occurs the vertebral body 4 on the concave aspect of the curve increases in size while the convex aspect is unable to grow as much.

This results in correction of the coronal plane deformity at the instrumented segment and also opening or change in angulation of the hinge 2 driven by growth.

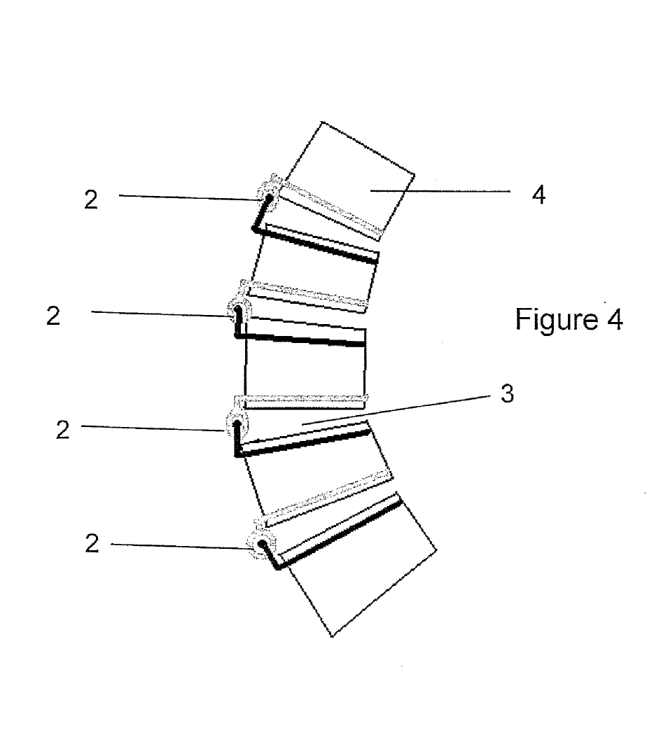

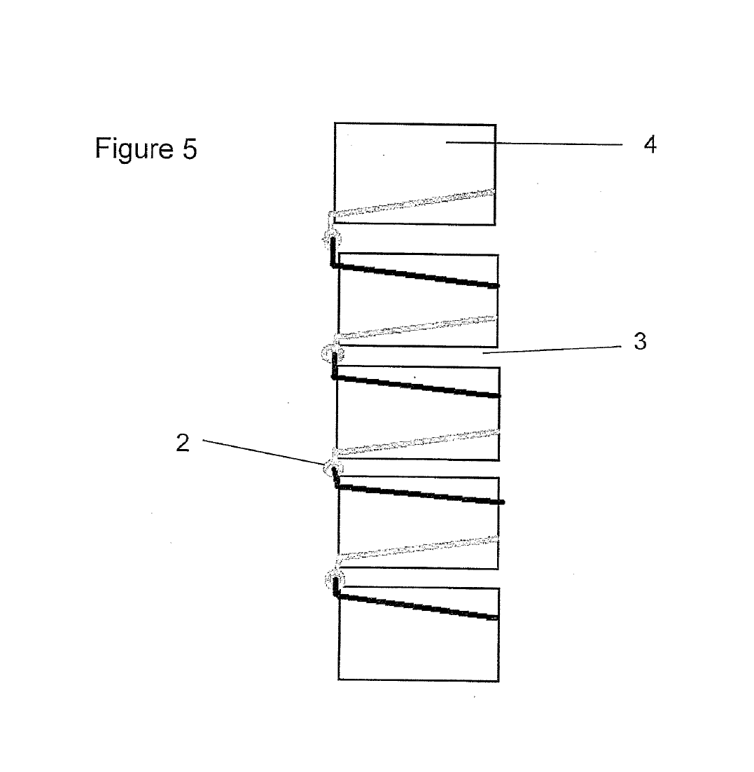

As illustrated in FIG. 4, multiple devices 1 may be applied on the convexity of a scoliosis deformity. As growth occurs, the hinges 2 open resulting in correction of the spinal deformity. As shown in FIG. 5, the spinal deformity is corrected after sufficient growth has occurred. The devices 1 may be connected together to form a single large construct with multiple attachment members for attaching the devices to vertebrae.

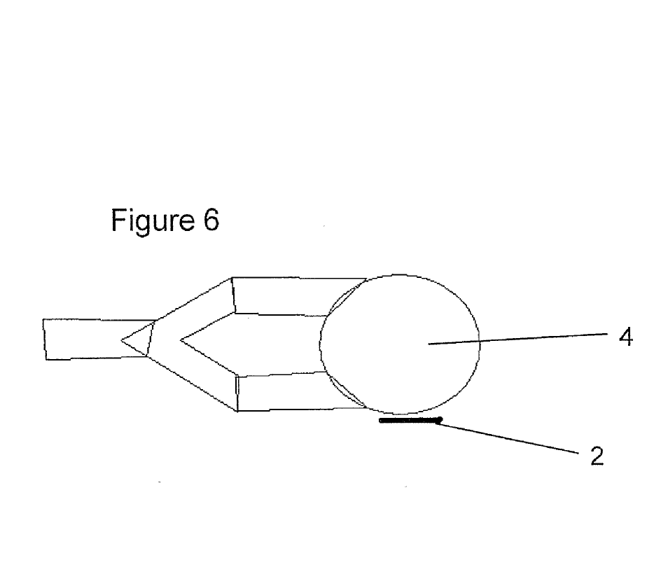

As illustrated by FIGS. 6 to 8, the angle of the hinge with respect to the anteroposterior axis of the spine can be modified in order to allow growth in a particular direction or directions.

The hinge 2 may be applied in a direct lateral position in which it will only give a coronal plane correction by virtue of its tethering and opening effect. As shown in FIG. 6, the hinge 2 of the device 1 is positioned parallel to the anteroposterior axis of the spine and therefore as the hinge opens with growth it will not affect the anteroposterior/sagittal contour of the spine.

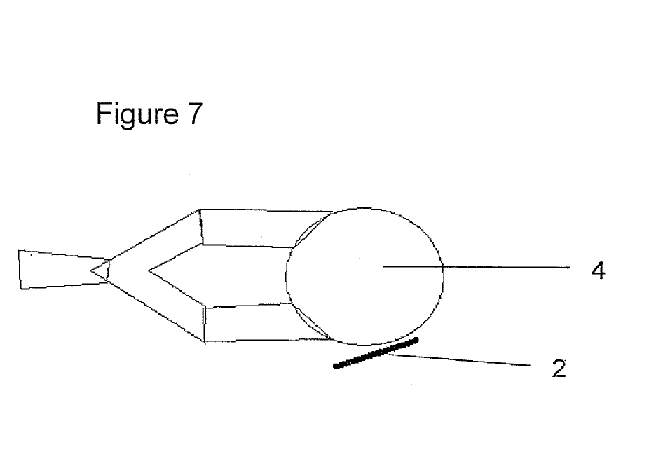

If the hinge is angled so that the medial aspect of it faces towards the posterior aspect of the vertebra then it will give coronal plane correction with an element of segmental kyphosis as it is opened by growth. As shown in FIG. 7, the hinge device is angled anteriorly to the anteroposterior axis of the spine and so as the hinge opens with growth it will have a kyphosing effect on the anteroposterior/sagittal contour of the spine.

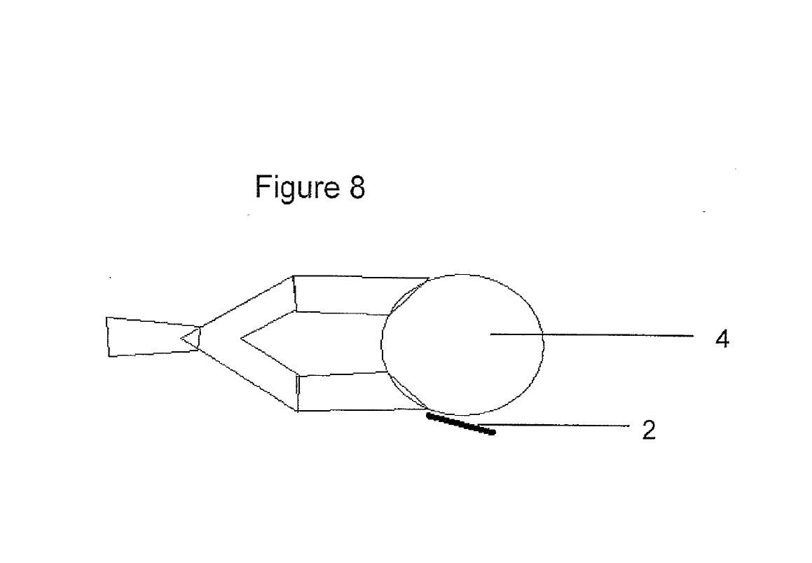

If the hinge is angled so that the medial aspect of it faces somewhat towards the anterior aspect of the vertebral body as it is opened by growth it will have an effect in both the coronal and saggittal planes, resulting in a degree of increased segmental lordosis. As shown in FIG. 8, the hinge device is angled posteriorly to the anteroposterior axis of the spine and so as the hinge opens with growth it will have a lordosing effect on the anteroposterior/sagittal contour of the spine.

If the hinge is angled in the vertical plane with regard to the horizontal axis of the growth plate then it can correct rotational deformity as it is opened by growth.

The hinge can be arranged in a “sloppy” format to allow some movement in all directions to reduce implant stresses and keep the spine tissues healthy. The sloppy hinge format may be configured to allow more movement in certain directions than others. The hinge can have a rising butt element incorporated to alter the centre of rotation induced by the hinge to a desired locus. Thus a hinged implant can give a three dimensional correction of a scoliosis.

The position of the hinge when it is attached to the spine has an effect on the amount of longitudinal growth it will permit on the side of the spine to which it is applied. This is an important consideration as not much growth is required in an adolescent whereas in a young child much more growth is required. As a hinge opens from a position where both components of the hinge are parallel the vertical distance travelled per degree of opening between the ends of the hinge components varies. Initially this distance is quite large but as the hinge opens further this distance diminishes until after the hinge has opened 180 degrees the distance starts to narrow again. Another factor affecting the longitudinal distance travelled between the hinge ends is the length of the hinge components. The longer a given hinge component the more distance it will travel per degree of opening.

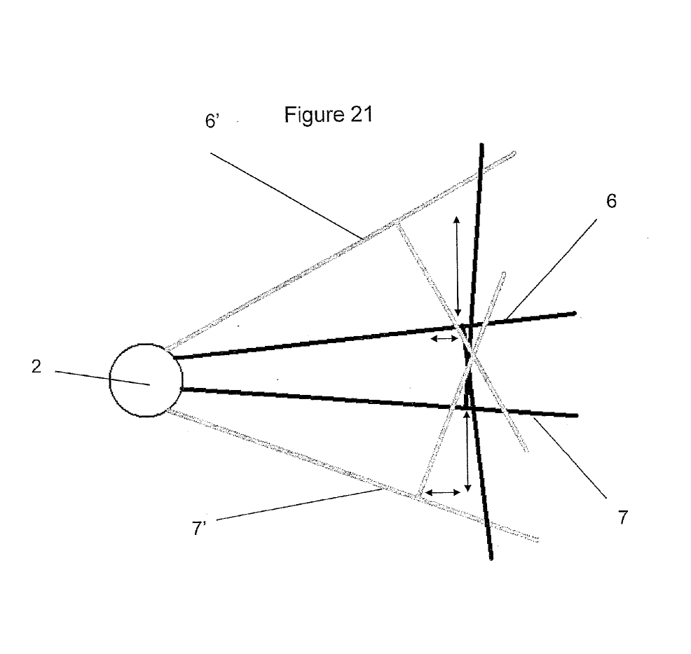

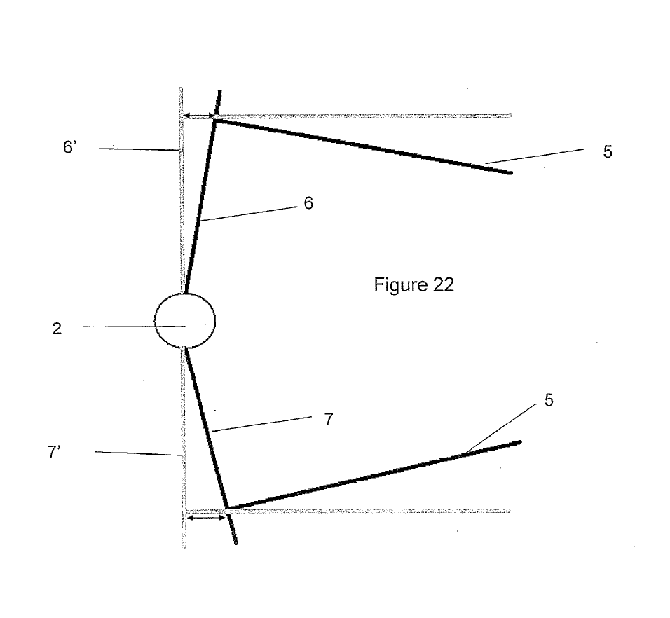

FIGS. 21 and 22 show the effect of opening a hinge on distance between the superior and inferior hinge components. If the hinge components are almost parallel to each other then much more longitudinal motion occurs as the hinge opens than if the hinge components are almost aligned end to end. Thus a hinge can be configured to give the amount of longitudinal growth required on the basis of the child's predicted growth. This will prevent over-correction of the deformity by the device.

As our knowledge of the mechanical stimuli that modulate growth is currently primitive the device 1 may need to be configured quite differently to the arrangement described.

The goal of treatment is to grow the spine back towards a normal shape. Dynamically loading a growth plate stimulates growth. It will be best to apply the device 1 in a way which maximally stimulates growth in the area of the vertebral body/posterior elements which are hypoplastic. This strategy is superior to one in which one attempts to use the device to force the spine to grow in the desired direction by harnessing the energy of its longitudinal growth to twist the spine deformity in a corrective direction. The latter strategy remains vastly superior to current methods of treatment where the deformity is allowed to progress until the child is old enough for surgery and then the spinal segments involved are fused with partial correction of the deformity.

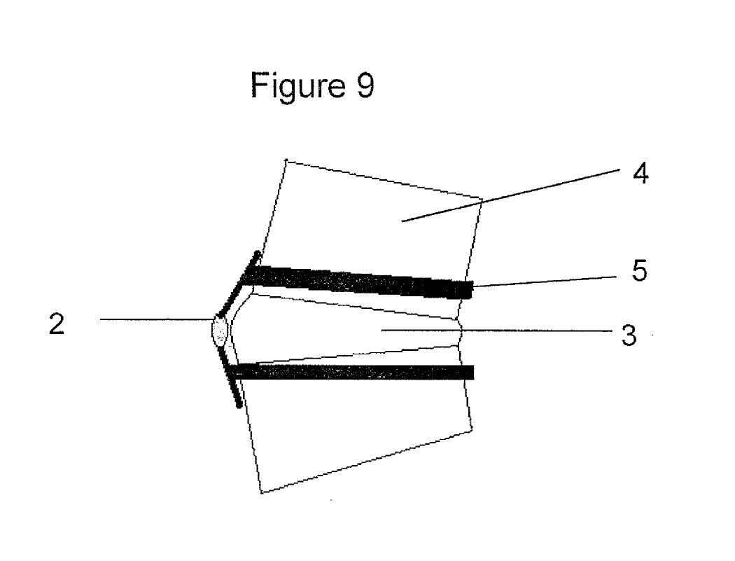

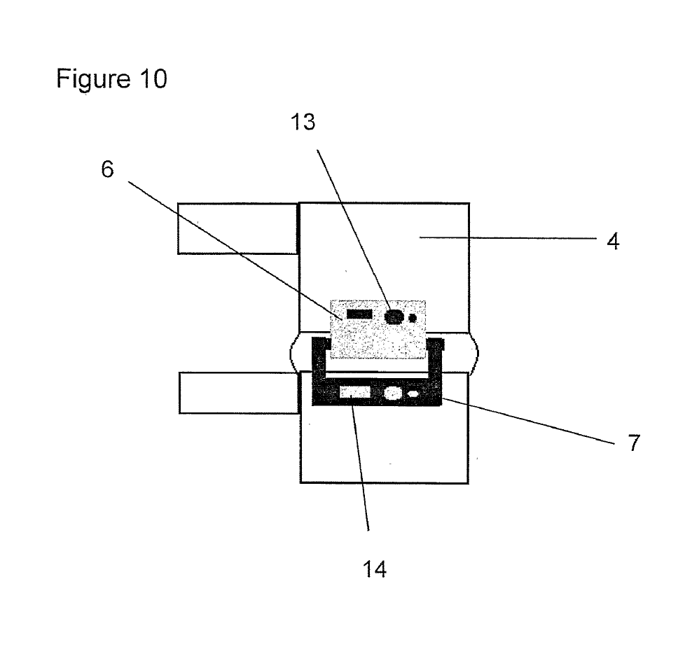

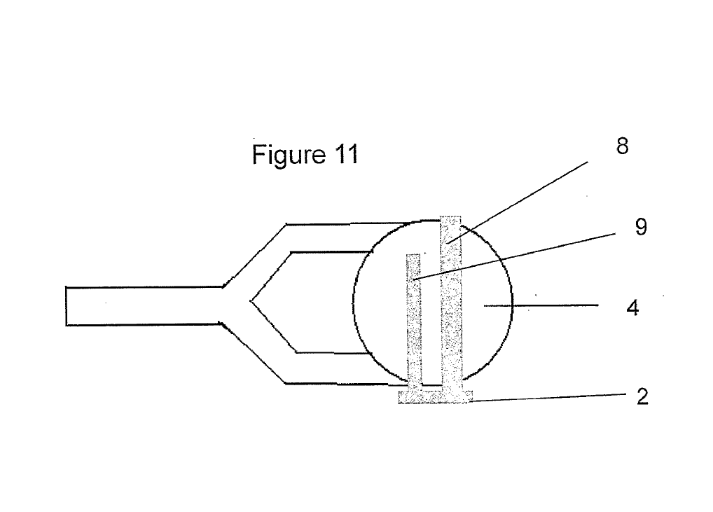

An embodiment of the hinge 2 is illustrated in FIGS. 9 to 11. The hinge 2 has a superior hinge component 6 and an inferior hinge component 7, each of which has adaptations 13, 14 for vertebral body attachment members 5. The attachment members 5 consist of one bone screw 8 coated with hydroxyapatite and a blade plate 9.

The screw 8 has threads on its head which screw into the adaptations 13, which may be in the form of threaded holes on the superior hinge component 6 and inferior hinge component 7. The hinge screw adaptations 13 have a further space for screws to lock the aforementioned screw heads into the adaptations on the hinge to prevent screw backout.

The blade plate 9 consists of a standard blade (of the type found in orthopaedic blade plates or staples but of appropriate size) and the plate component of the blade plate is the hinge adaptation 14 for vertebral body attachment members.

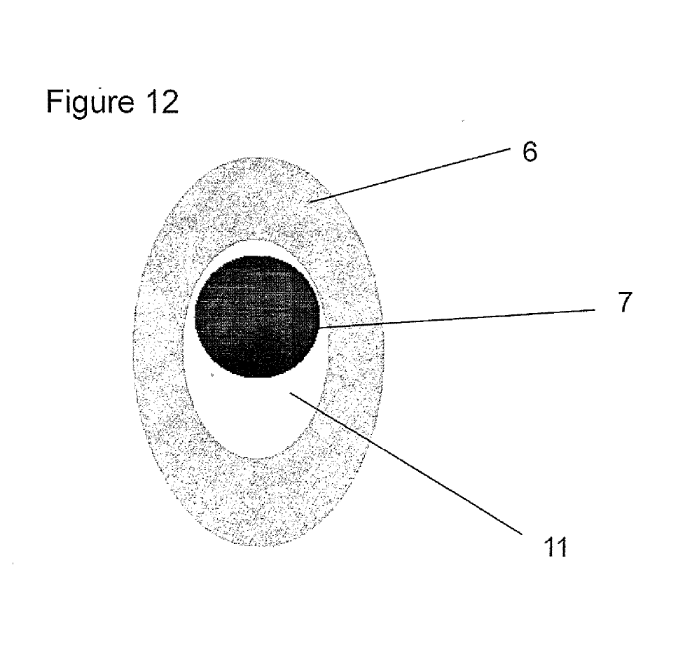

The superior hinge component 6 and inferior hinge component 7 articulate with a tolerance/clearance allowing some flexion extension, rotation and lateral flexion to occur at the hinge bearing 11. This tolerance is elliptical in shape, as shown in FIG. 12, to allow the hinge 2 to narrow somewhat as the spine moves in a direction which acts to compress the hinge.

There is room for movement to a limited extent in all directions helping reduce implant stresses and maintain tissue health. In particular there is room for the hinge to shorten as the spine moves in a corrective direction under the influence of the patient's muscles. Allowing this movement reduces the stresses on the device 1. The hinge bearing surfaces have sloped edges 12 to prevent pinching of tissue as the hinge 2 moves.

The hinge 2 may be angled in the vertical plane perpendicular to the growth plate which occupies the horizontal plane for the purposes of this description.

In this situation opening of the hinge 2 as growth occurs, results in both rotational and coronal plane deformity correction. The hinge 2 angulation causes the superior hinge component 6 and inferior hinge component 7 to rotate relative to one another through two orthogonal planes as the hinge is opened or closed. The hinge 2 angulation in this plane is selected to treat the particular deformity. The greater the amount of angulation in this (vertical) plane the more derotation that will occur relative to coronal plane correction.

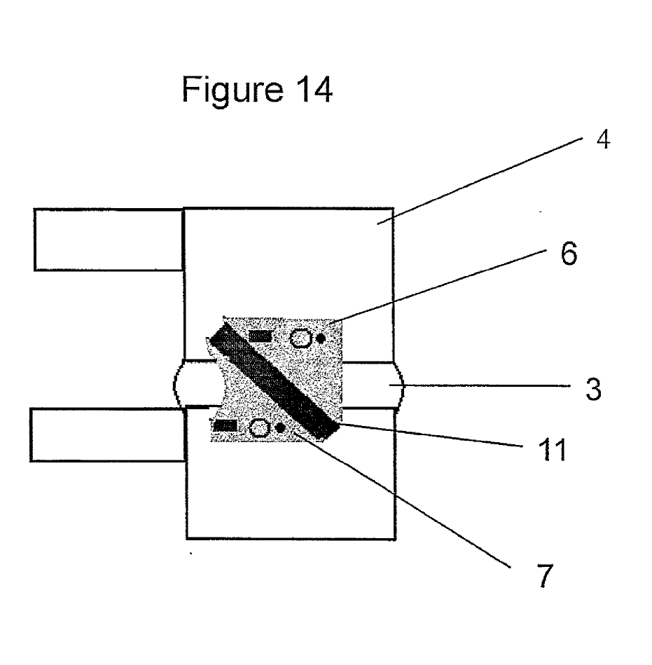

As shown in FIG. 14, the hinge 2 is orientated at approximately 45 degrees vertically to the horizontal plane, the anterior aspect of the hinge being inferior to the posterior aspect. As growth occurs the hinge will open resulting in both a coronal and a rotational correction of the deformity at that motion segment. This configuration of the apparatus 1 is appropriate for correction of rotational deformity in motion segments located superior to the apex of a right sided scoliosis.

If the hinge 2 is placed at 90 degrees to the plane of the growth plate (horizontal plane), pure derotation will occur with no coronal plane correction as the hinge opens. As the driving force for hinge opening is in the vertical plane the hinge angle must be less than 90 degrees to allow this force to open it however the hinge angle may approach 90 degrees in cases where severe rotational deformity is present. Similarly, the hinge angle must be greater than but may approach 0 degrees where only a small rotational deformity is present.

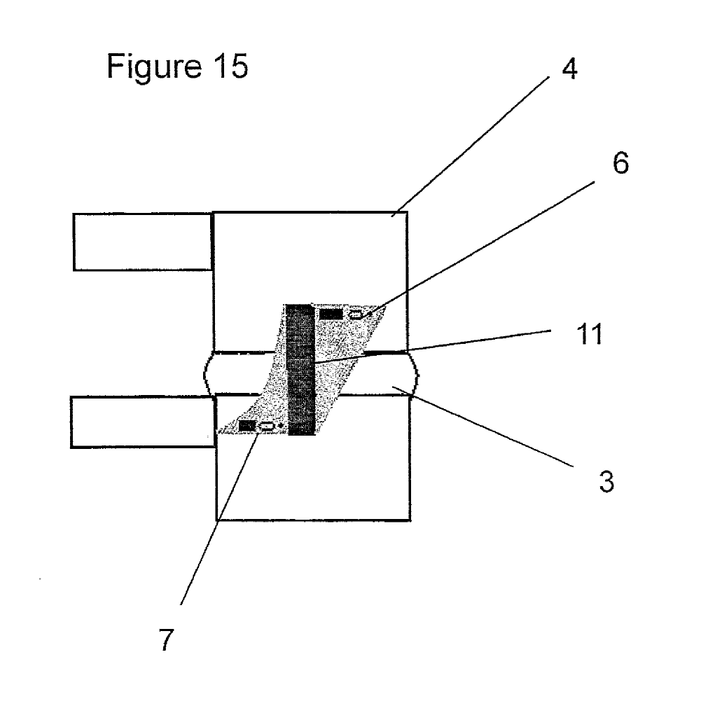

As illustrated in FIG. 15, the hinge is orientated at just less than 90 degrees vertically to the horizontal plane, the anterior of the hinge being inferior to the posterior aspect. As growth occurs the hinge will open resulting in an almost purely rotational correction of the deformity at that motion segment.

The hinge angulation in the vertical plane is in a different direction depending on the location of the hinge in the curve. The point at the posterior end of the hinge bearing 11 is considered as the point about which the hinge is angled. In a right sided curve the segments superior to the apical vertebra will have appropriate derotation when the anterior aspect of the hinge 2 is angled inferiorly with regard to the posterior aspect of the hinge (FIG. 14). In a right sided curve the segments inferior to the apical vertebra will have appropriate derotation when the anterior aspect of the hinge is angled superiorly with regard to the posterior aspect of the hinge. If the hinge is placed parallel to the growth plate then correction occurs only in the coronal plane as the hinge 2 opens ie there would be no rotational correction.

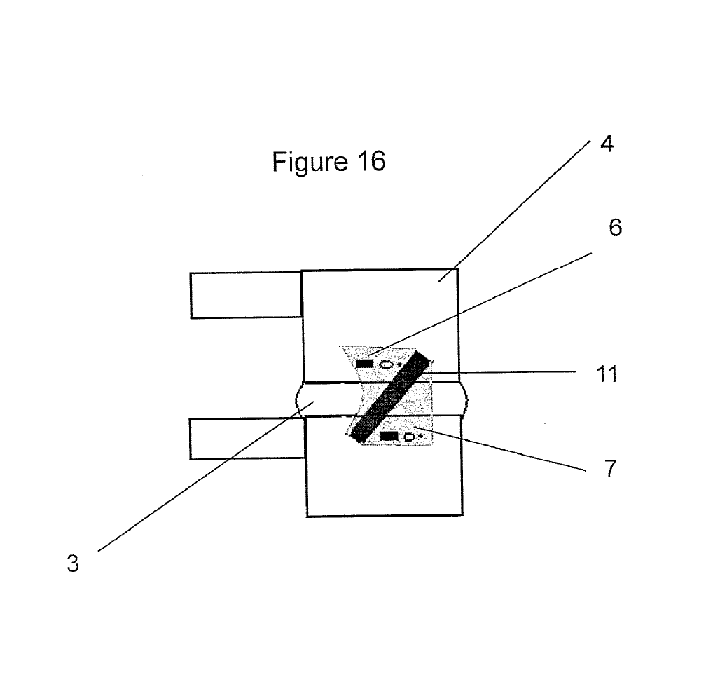

FIG. 16 shows an embodiment of the apparatus 1 attached to the lateral aspect of the spine on the convexity of the curve. The hinge is orientated at approximately 45 degrees vertically to the horizontal plane, the posterior aspect of the hinge being inferior to the anterior aspect. As growth occurs the hinge 2 will open resulting in both a coronal and a rotational correction of the deformity at that motion segment. The device is appropriate for correction of rotational deformity in motion segments located inferior to the apex of a right sided scoliosis.

The sagittal profile is controlled by angulation of the hinge 2 relative to the anterior posterior axis of the growth plate 3. If the hinge 2 is angled parallel to the anteroposterior axis (as shown in FIG. 6) then the sagittal profile will be unchanged. If it is angled anteromedially (as shown in FIG. 7) then as the hinge 2 is opened by growth it will increase the amount of kyphosis present. If the hinge is angled posteromedially (as shown in FIG. 8) then as it opens with growth it will increase the amount of lordosis present.

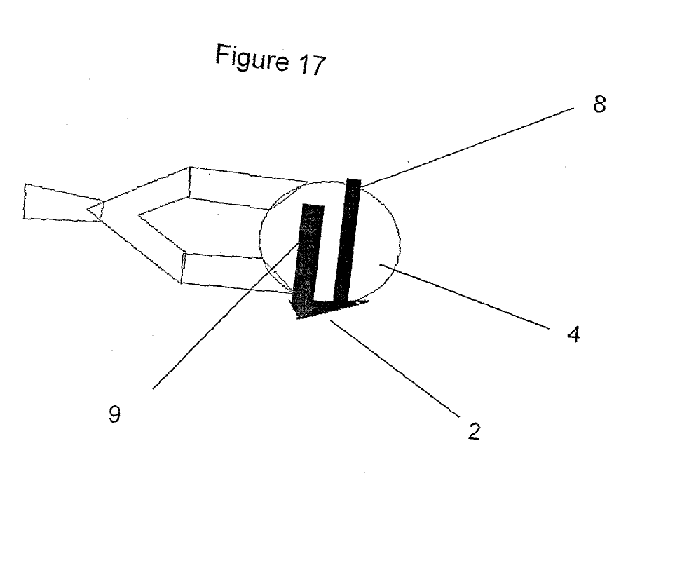

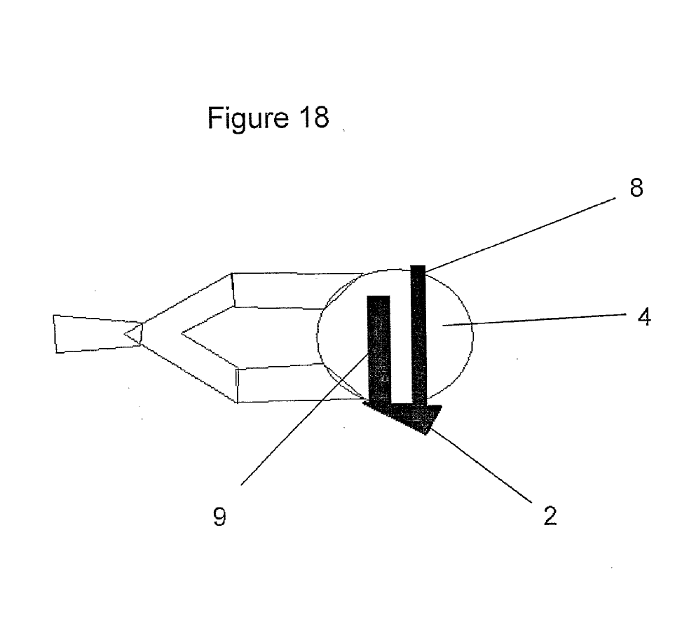

The posterior aspect of the hinge may be scalloped to allow for placement of the hinge without resecting the rib head which lies over the posterior part of the disc, as viewed from inside the chest. The superior hinge component 6 and inferior hinge component 7 may be modified to allow a better fit of the apparatus 1 to the spine according to its configuration. Thus a kyphosing apparatus 1 as shown in FIG. 17 may be shaped somewhat differently to a lordosing apparatus 1 as shown in FIG. 18.

FIG. 17 shows an embodiment of the implantable apparatus attached to the lateral aspect of the spine on the convexity of a curve. The hinge 2 is orientated at approximately 15 degrees anterior to the anteroposterior axis of the spine. Thus as the apparatus 1 is opened by growth it will have a kyphosing effect on the spine.

FIG. 18 shows an embodiment of the implantable apparatus attached to the lateral aspect of the spine on the convexity of a curve. The hinge 2 is orientated at approximately 15 degrees posterior to the anteroposterior axis of the spine. Thus as the apparatus 1 is opened by growth it will have a lordosing effect on the spine.

There are a range of embodiments of the apparatus to correct different deformities thus allowing treatment tailored to the individual deformity to optimise correction as growth occurs.

The amount of growth potential remaining in a deformed spinal segment to be instrumented with the device 1 may affect the configuration of the apparatus.

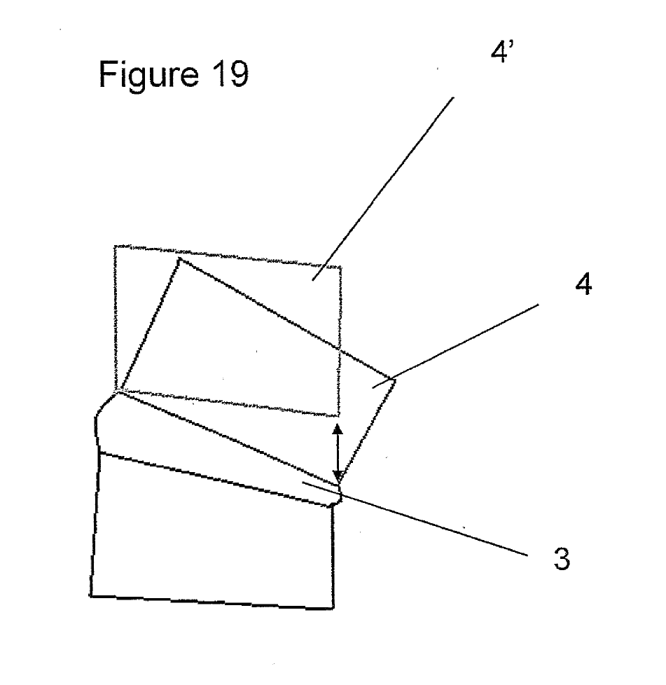

It is important to allow growth of the convexity of the curve in addition to the concavity otherwise in the younger child with higher residual growth potential it may be possible to achieve over-correction of the curve. This concept is particularly important if treating a young child with a relatively small curve. In a given deformity as the curve is corrected by growth most of the growth occurs on the concavity of the curve. The concave aspect of the spine has several years of growth potential to make up before the spine will start to overcorrect. FIG. 19 shows an anterior view of a spinal motion segment in a scoliosis deformity. The vertebral body 4′ denotes the position of the superior vertebral body of the motion segment relative to the inferior vertebral body after growth has corrected the concave deformity but without any growth having occurred on the convexity. It is clear that a considerable amount of growth is required to occur on the concavity of the scoliosis deformity before growth on the convexity is necessary to allow spinal lengthening without curve over-correction.

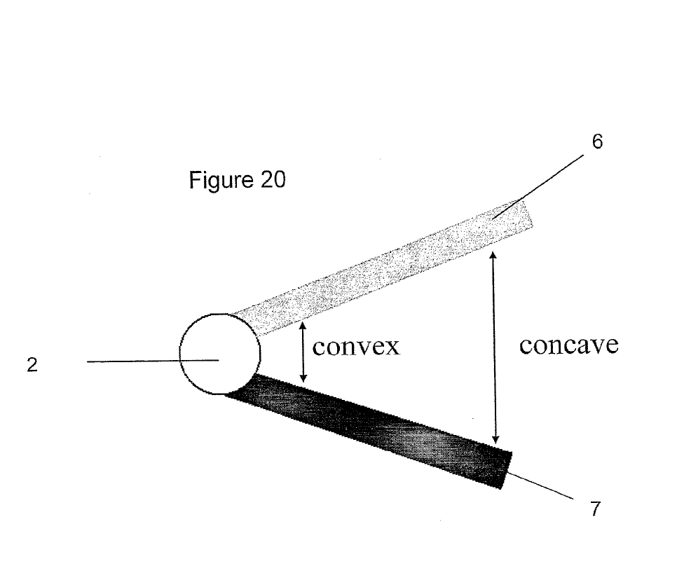

FIG. 20 shows that as the hinged device is pushed open by growth there is potential for some growth to occur on the convexity of the deformity but that this is much less than the amount of growth which occurs on the concavity of the deformity.

In the embodiment shown in FIG. 20, the distance travelled by the points corresponding to the concavity of the curve is much greater than that corresponding to the convexity of the curve.

The extent of growth permitted on the convexity of the curve as the hinge opens can be varied. This may be done by having a variety of implants 1 with the hinge being open to different extents at time of implantation in each.

FIGS. 21 and 22 illustrate the effect of the initial hinge position (at implantation) on the amount of longitudinal growth permitted by the device 1.

As shown in FIG. 21, the superior and inferior hinge components 6,7 are approximately parallel at implantation. After growth occurs the hinge opens and the superior and inferior hinge components 6,7 move to the positions 6′,7′. The amount of lengthening is considerable for the angular degree of opening of the hinge 2 that has occurred. As the hinge 2 opens from a position where the two limbs of the hinge are parallel maximal lengthening (in a longitudinal direction) per degree of opening occurs.

When the superior and inferior hinge components 6,7 of the hinge 2 are aligned at almost at 180 degrees minimal lengthening per degree of opening occurs (as shown FIG. 22). This is because the initial movement from a position where the hinge limbs are parallel is almost fully in a longitudinal direction whereas the final movements towards alignment of the hinge limbs at 180 degrees to each other are almost perpendicular to the longitudinal axis. Thus to get the maximum angular correction from a small amount of residual growth (growth causes longitudinal length change) one would want a configuration where the hinge components were almost aligned.

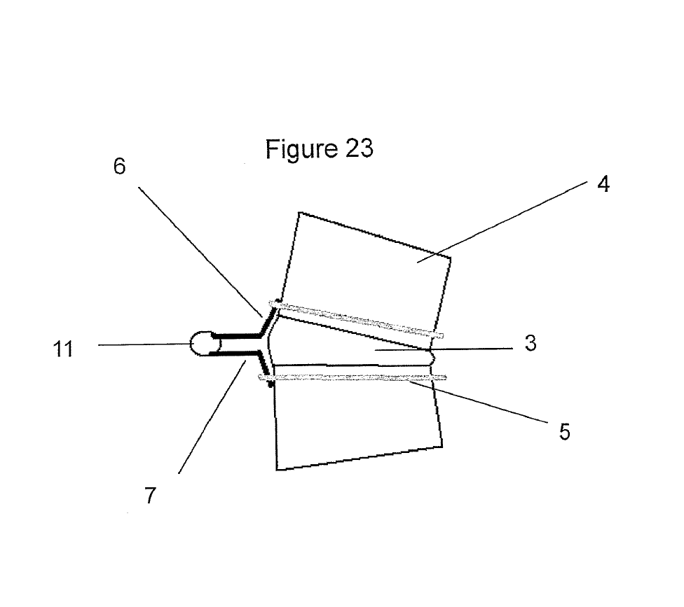

Conversely in order to allow growth to occur at a spinal segment one would favour an arrangement where the limbs of the hinge (superior and inferior hinge components 6,7) were more parallel at the start of treatment. An example is shown in FIG. 23, where the device is designed to facilitate lengthening of the convexity of the curve as growth occurs, thus permitting more longitudinal growth of the instrumented motion segment to prevent curve over-correction when treating young children.

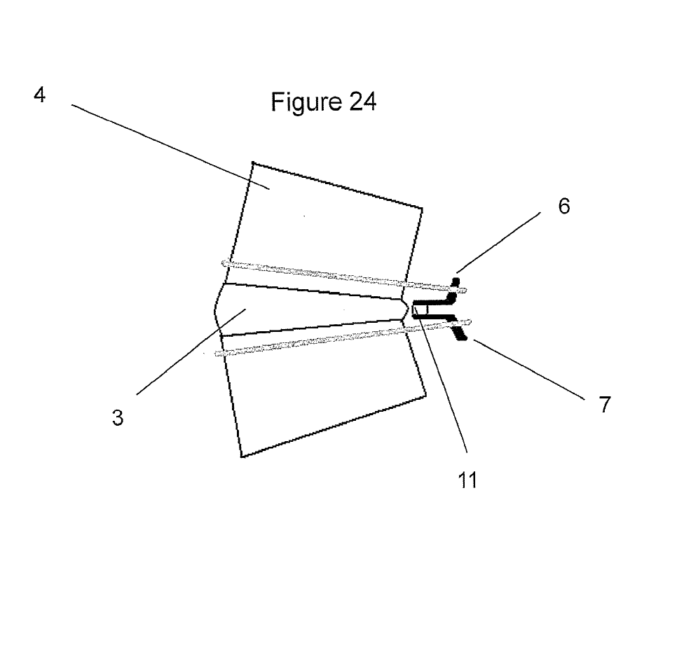

Certain embodiments of the device 1 may also be applied to the concave aspect of a curve. This may be necessary for example in the treatment of curves thoracoscopically in small children with big curves such as in cerebral palsy. This approach would also be useful in those who had failure of a device inserted on the convexity as it would allow salvage of the situation without having to re-operate on the curve convexity thus reducing the potential for complications. FIG. 24 shows an anterior view of a spinal motion segment in a scoliosis deformity with an embodiment of the hinge device attached on the concavity of the curve. This device is designed to facilitate lengthening of the concavity of the curve as growth occurs while preventing growth on the convexity. It will allow instrumentation of the concavity for revision cases and those where this is technically less demanding/required.

In those with minimal remaining growth potential the device may act to compress the convexity of the curve as growth occurs thus helping reshape the spine by reducing the extent of disc wedging. The device will act in this way if the hinge limbs are at 180 degrees to each other when it is implanted. As it is opened by growth it will shorten on the convexity while lengthening on the curve concavity.

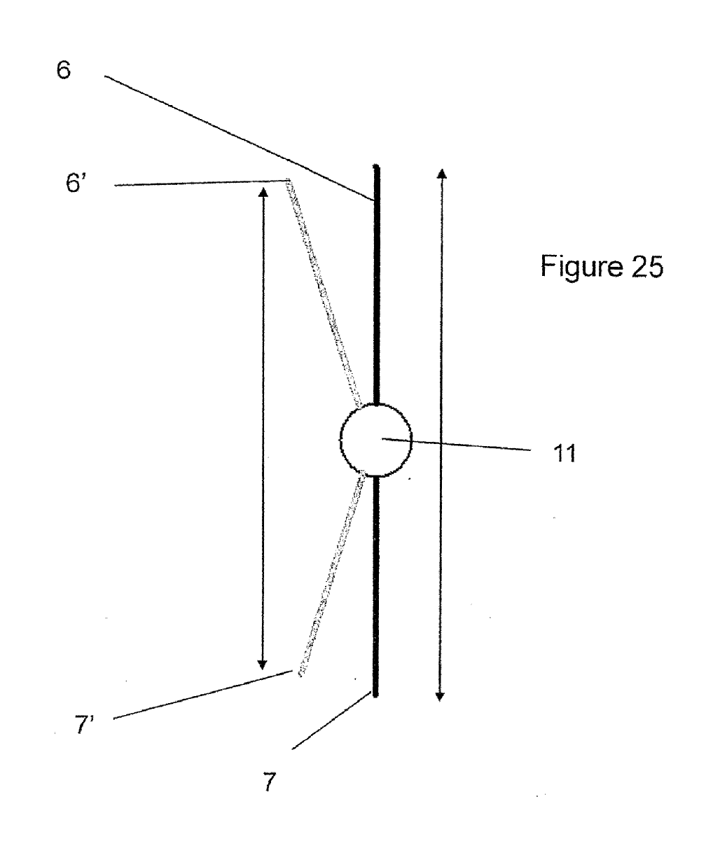

FIG. 25 shows a hinge viewed from the anterior aspect and illustrates the effect of initial hinge position on amount of longitudinal growth permitted by the device. The position of the hinge limbs 6,7 and vertebral attachment members 5 are shown as at the time of device implantation.

The position of the hinge limbs 6′,7′ and vertebral attachment members 5′ some time after device implantation when growth has opened the hinge is illustrated. The hinge 2 has shortened which would result in compression on the convexity of a scoliosis to which it was applied.

The hinge 2 may have a constraint built in so that it will not close beyond a certain point thereby acting to offload the concave aspect of the curve. The device may alternatively or additionally have a dynamic offloading element such as a cable or biasing means such as a spring incorporated to facilitate maximal growth stimulation and hence maximal curve correction especially in cases where less than optimal residual growth remains.

With reference to the inclusion of a dynamic offloading element in the implantable device, such an element allows forces, and in particular dynamic forces, to be exerted on the skeletal structure which it would not normally experience. With no dynamic offloading element the skeletal structure would experience dynamic loads by virtue of changing muscle forces as the person moves about. However, it is thought that growth at a growth plate may be encouraged by stimulating the growth plate in a manner that is different to the stimuli it is conditioned to receiving. If a dynamic offloading element, such as a spring, is included in the implant, a part of the dynamic load normally experienced by the skeletal structure will be transferred to and stored in the spring. As the person moves about the energy stored in the spring will be released through the implant and hence those parts of the skeletal structure to which it is attached, thereby loading that structure in a different manner to that which it is conditioned to receive.

The implantable device may include a biasing means to exert a substantially static load. For example, in the apparatus illustrated in FIG. 4, a biasing means may be provided to exert a load on the vertebrae which reduces the load on the concave side thereof.

The device may be made from a variety of materials but stainless steel or cobalt chrome are the best candidates. The exact dimensions of the device will be determined by design considerations and the need to prevent fatigue failure.

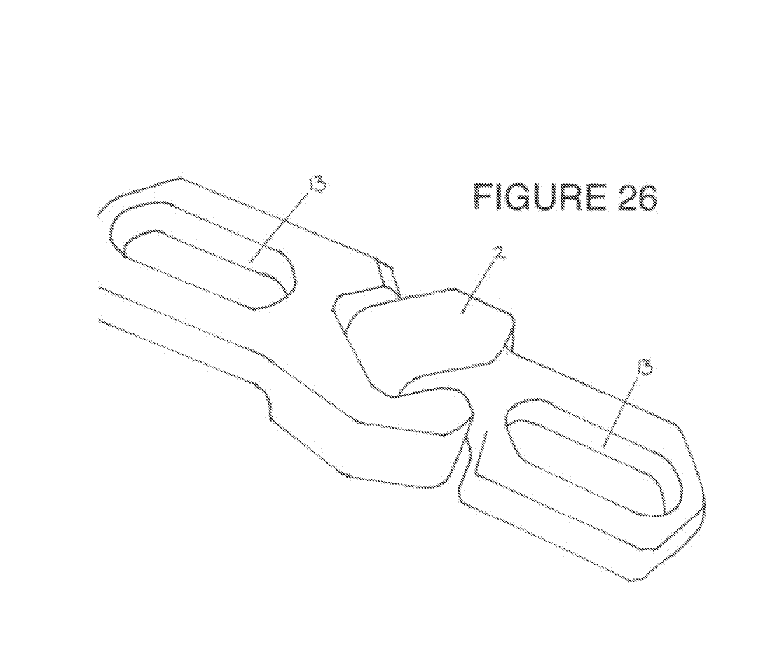

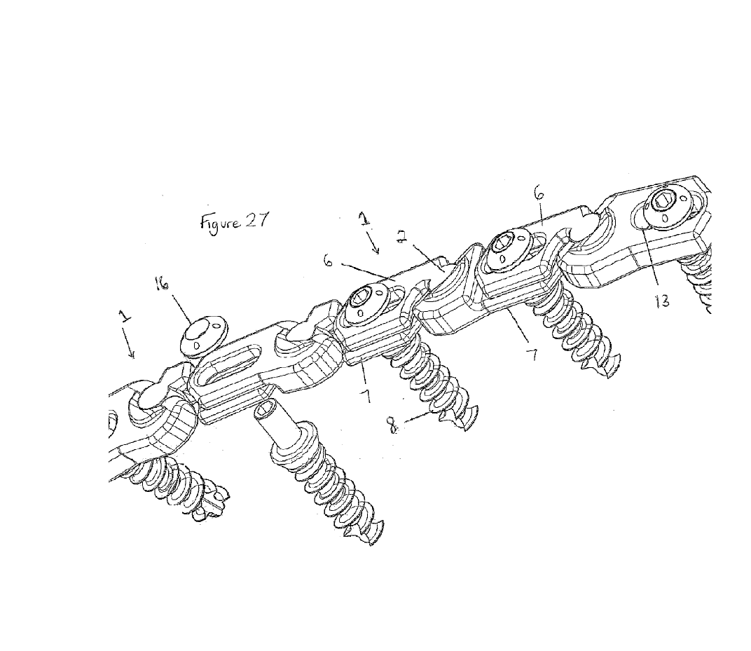

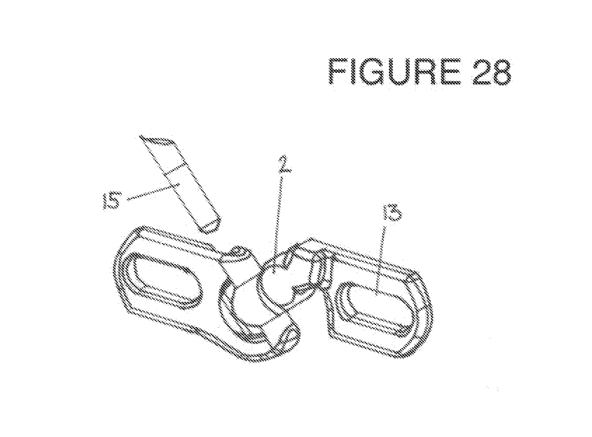

FIGS. 26 shows alternative embodiments of the hinge 2 of the device 1. FIGS. 27 and 28 show the preferred embodiments of the device, having a ball and socket type hinge in which movement is restricted by a hinge pin 15.

In an alternative embodiment the ball and socket hinge may allow full rotational movement.

FIG. 27 shows a preferred embodiment of the apparatus comprising more than one device 1, joined to form a single construct.

As illustrated in FIGS. 27 and 28, attachment members 8 in the form of bone screws are shown located in adaptations 13, which in this embodiment are slots. The adaptations 13 are shaped such that the position of the bone screw in the adaptation or slot may be varied when fitting the device, to allow for differences in the size or length of patients' spines. FIG. 27 shows how the attachment members 8 are fitted through adaptations 13 in the devices 1 so that hinge component 6 is linked to an adjacent hinge component 7 of another device 1, to form a single construct. A screw cap 16 is screwed down onto the bone screw/attachment member 8 to hold a device 1 in position and also to fasten together hinge components 6,7 of adjacent devices 1.

Surface coatings may be applied to the device 1. For example, a rough textured surface coating may be applied in the region where the screw cap 16 is fitted to the device, which will help to hold the hinge components together and prevent the screw cap 16 from turning. A smooth surface coating may be applied at the ball and socket type hinge to facilitate opening or closing of the hinge. The smooth coating may be a carbon based coating, which provides very low friction and good wear properties.

In use, each attachment member 8 is screwed into a verterbra and each hinge mechanism is located between two adjacent vertebrae.

The device is suitable for use in the treatment of adults as well as children.

To apply the apparatus to a patient, the patient is positioned for a thoracoscopic approach to the spine. The levels to be instrumented are selected as one would do for conventional scoliosis corrective surgery. The patients curve will be partly corrected by their position on the operating table. The device 1 is applied to the deformed spine and does not alter the position of the spine at the time of application thus minimising the risk of inducing neurologic dysfunction. The device is inserted in the same manner as a staple. As with other stapling techniques for treatment of scoliosis the segmental vessels are not taken thus reducing the risk of neurological injury.

The spine is exposed anteriorly. The hinge limbs 6,7 have screw holes which have threads for the head of a screw similar to that used in a locking plate. An instrument is used to simultaneously cut a slot on either side of the disc to be instrumented to receive the blade plates of the hinge limbs. A hole is then drilled adjacent to the slots for the blade plates using drill guide slots in the instrument used to cut the blade plate slots. This instrument is then removed and the implant positioned using another instrument to hold the hinge in the appropriate position for insertion onto the spine. The hinge is tapped down to drive the blade plates down the pre-prepared slots. The screws are then inserted via the guides on the insertion instrument and tightened. The locking screws are then inserted to prevent screw backout. The next level to be instrumented is selected and the process is repeated.

Post-operatively the patients would be expected to mobilise quickly and be discharged within a few days. The pain from a thoracoscopic procedure is much less than that of thoracotomy or fusion and the blood loss is usually minimal. The device may be used at all levels in the spine and the surgical approach would vary according to which vertebrae were to be instrumented.

The apparatus 1 can provide a degree of mobility of the tissues between its attachment members while effecting a three dimensional correction of deformity without over-correction using a low profile construct. This invention may be adapted to treat spinal deformity from a posterior approach. Kyphotic deformities would be most suitable for posterior instrumentation with this device but any spinal deformity could be treated.

Rather than comprising a pair of hinge limbs 6, 7 which are adapted for attachment to parts of a skeletal structure, one or both of the hinge limbs 6, 7 may be adapted to engage with a part of a skeletal structure without being attached thereto. For example, one or more of the hinge limbs may be a flat plate abutting a bone, without screws passing through the hinge limb and into the bone. This situation would arise if the hinge was placed in a disc space as a disc replacement device. The hinge limbs 6, 7 would be contoured appropriately to interact with the intervertebral disc endplates and the hinge would be present between the limbs allowing a controlled range of motion between the vertebral bodies. Other adaptations of the device thus described may be amenable for use in joint replacement surgery at other sites than the intervertebral disc such as the elbow and ankle.

While the present invention has been shown and described in terms of preferred embodiments thereof, it should be understood that this invention is not limited to any particular embodiment and that changes and modifications may be made without departing from the true spirit and scope of the invention.

1. An implantable skeletal growth deformity correction device, the device comprising:
a hinged element including a hinge which lies on a hinge axis; and
engagement means disposed to either side of the hinge axis of the hinged element and adapted to engage with a skeletal structure on either side of a growth plate and position the device with respect to the skeletal structure;
wherein the hinge lying on the hinge axis is adapted to generate relative rotational motion between the engagement means disposed to either side of the hinge axis through two orthogonal planes upon opening and closing of the hinge about the hinge axis; and
wherein the hinge axis lies in a first plane that, in use with the device implanted in the body of a patient, is perpendicular to a plane of the growth plate, and wherein in the first plane the hinge axis lies on a second plane, the second plane lying at an angle of more than 0 degrees and less than 90 degrees with respect to the plane of the growth plate.

2. An implantable skeletal growth deformity correction device as claimed in claim 1, wherein an element of the engagement means disposed to one side of the hinged element comprises an attachment member adapted to attach to a skeletal structure.

3. An implantable skeletal growth deformity correction device as claimed in claim 2, wherein the engagement means disposed to either side of the hinged element each comprise an attachment member adapted to attach to a skeletal structure.

4. An implantable skeletal growth deformity correction device as claimed in claim 1, wherein, in use the extent of opening of the hinged element changes in response to movement of the skeletal structures on either side of the hinged element to which the device is attached.

5. An implantable skeletal growth deformity correction device as claimed in claim 4, wherein the hinged element is adapted to encourage a desired movement of the skeletal structure on the at least one side of the hinged element.

6. An implantable skeletal growth deformity correction device as claimed in claim 1, further comprising a constraint element, wherein the constraint element restricts opening or closing of the hinged element.

7. An implantable skeletal growth deformity correction device as claimed in claim 6, wherein the constraint element is adjustable.

8. An implantable skeletal growth deformity correction device as claimed in claim 1, wherein the hinged element allows movement of skeletal structures in more than one direction.

9. An implantable skeletal growth deformity correction device as claimed in claim 8, wherein the hinged element allows movement of skeletal structures in more than one dimension.

10. An implantable skeletal growth deformity correction device as claimed in claim 1, wherein the hinged element comprises a hinge pin.

11. An implantable skeletal growth deformity correction device as claimed in claim 1, wherein the hinged element comprises a sloppy hinge.

12. An implantable skeletal growth deformity correction device as claimed in claim 2, further comprising an aperture, and wherein the attachment member locates in the aperture.

13. An implantable skeletal growth deformity correction device as claimed in claim 12, wherein the aperture is threaded.

14. An implantable skeletal growth deformity correction device as claimed in claim 12, wherein the aperture is an elongate slot.

15. An implantable skeletal growth deformity correction device as claimed in claim 1, further including a biasing means, arranged to exert a force on the device to cause motion thereof about the hinge element.

16. An implantable skeletal growth deformity correction apparatus comprising a plurality of implantable skeletal growth deformity correction devices as claimed in claim 1.

17. An implantable skeletal growth deformity correction apparatus as claimed in claim 16, wherein adjacent implantable skeletal growth deformity correction devices are configured to be connected together.

18. An implantable skeletal growth deformity correction apparatus as claimed in claim 17, wherein adjacent implantable skeletal growth deformity correction devices are adapted to be connected together by an attachment member.

19. An implantable skeletal growth deformity correction apparatus as claimed in claim 16, wherein the implantable skeletal growth deformity correction devices are adapted to be connected together by a connecting element.

20. An implantable skeletal growth deformity correction device as claimed in claim 1, wherein the hinged element includes a ball and socket, the ball and socket providing the hinge.

21. An implantable skeletal growth deformity correction apparatus as claimed in claim 16, wherein the hinge of the hinged element of one implantable skeletal growth deformity correction device lies on a hinge axis that is a mirror image of the hinge axis of a hinged element of another of the implantable skeletal growth deformity correction devices of said plurality of implantable skeletal growth deformity correction devices.
